(19) United States
(12) Patent Application Publication
(10) Pub. No.: US 2009/0328250 A1
(43) Pub. Date: Dec. 31, 2009

(54) EXPRESSION OF TGF-BETA IN PLASTIDS

(75) Inventors: Mark William James Ferguson, Manchester (GB); Hugh Gerard Laverty, Manchester (GB); Nick Occleston, Manchester (GB); Sharon O'Kane, Manchester (GB); Martin Gisby, Manchester (GB); Anil Day, Manchester (GB); Phillip Mellors, Manchester (GB)

Correspondence Address: GOODWIN PROCTER LLP;PATENT ADMINISTRATOR, 53 STATE STREET, EXCHANGE PLACE, BOSTON, MA, 02109-2881, US

(73) Assignee: Renovo Limited, Manchester (GB)

(21) Appl. No.: 12/440,688

(22) PCT Filed: Sep. 11, 2007
(86) PCT No.: PCT/GB07/03416

§ 371 Date: Mar. 10, 2009

(30) Foreign Application Priority Data
Sep. 11, 2006 (GB) 0617816.4

(51) Int. Cl. C12N 15/82; C07K 14/475; C12N 15/11; A01H 5/00

(52) U.S. Cl. 800/278; 530/399; 536/23.5; 800/298

(57) ABSTRACT

Provided is a method for the expression of a TGF-β in a plant. A chimeric nucleic acid sequence comprising: (1) a first nucleic acid sequence capable of regulating the transcription in a plant cell of (2) a second nucleic acid sequence, encoding a TGF-β, and adapted for expression in the plant cell; and (3) a third nucleic acid sequence encoding a termination region functional in said plant cell is introduced into a plant cell and the plant cell grown to produce TGF-β. The nucleic acid sequence may preferably be adapted for expression in a plant chloroplast. It is preferred that the TGF-β is TGF-β3, whether full length or in the form of an active fragment.

The present invention relates to the expression of Transforming Growth Factor-Betas (TGF-βs). The invention relates to expression of TGF-βs in plants. In particular the invention relates to the expression of TGF-βs in plant chloroplasts. TGF-β3 is a preferred TGF-β for expression in accordance with the invention. The invention also provides chimeric nucleic acid sequences suitable for use in the expression of TGF-βs in plants, as well as TGF-βs produced by such methods, and uses of such TGF-βs.

The TGF-βs are a family of cytokines having diverse biological activities. Five members of the TGF-β family have been identified to date, the isoforms TGF-β1, TGF-β2, TGF-β3, TGF-β4, and TGF-β5. These TGF-βs share structural similarities, such as a common cysteine knot motif, as well as common signal transduction pathways.

The TGF-βs have biological activities that are of utility in many different therapeutic contexts. As a result there is much interest in the pharmaceutical application of TGF-β family members.

To date, the greatest pharmaceutical interest has been shown in TGF-β1, TGF-β2 and TGF-β3. These isoforms, which are all found in humans, are known to play crucial roles in the regulation of the wound healing response.

TGF-β1 has uses in the prevention and/or treatment of scleroderma, angiogenesis disorders, renal disease, osteoporosis, bone disease, glomerulonephritis and renal disease.

TGF-β2 may be used in the treatment of glioma, non-small-cell lung cancer, pancreas tumour, solid tumours, colon tumour, ovary tumour, age-related macular degeneration, ocular injury, osteoporosis, retinopathy, ulcers, carcinoma, mouth inflammation and scleroderma.

TGF-β3 may be used in the treatment of fibrotic disorders, scleroderma, angiogenesis disorders, restenosis, adhesions, endometriosis, ischemic disease, bone and cartilage induction, in vitro fertilisation, oral mucositis, renal disease, prevention, reduction or inhibition of scarring, enhancement of neuronal reconnection in the peripheral and central nervous system, preventing, reducing or inhibiting complications of eye surgery (such as LASIK or PRK surgery).

Current methods for the production of TGF-βs, particularly for therapeutic use, rely upon the expression of these proteins (either in their active or proprotein form) by cultured animal cells or cultures of appropriately transfected bacteria. Although such methods are effective for the production of TGF-βs they tend to produce relatively low yields, and the costs involved in the preparation of such proteins are high.

In the light of the above, it will be recognised that there is a well defined need to develop new methods for the production of TGF-βs that are not subject to these disadvantages.

It is an aim of certain embodiments of the invention to overcome or obviate at least some of the disadvantages of the prior art. It is an aim of certain embodiments of the invention to provide methods and/or means that may be used in the manufacture of TGF-βs at a lower cost than the methods of the prior art. It is an aim of certain embodiments of the invention to provide methods and/or means that may be used in the manufacture of TGF-βs in greater quantities than can be manufactured using the methods of the prior art.

According to a first aspect of the invention there is provided a method for the expression of a TGF-β in a plant, said method comprising:

(a) introducing into a plant cell a chimeric nucleic acid sequence comprising:
(1) a first nucleic acid sequence capable of regulating the transcription and/or translation in a plant cell of
(2) a second nucleic acid sequence, encoding a TGF-β, and adapted for expression in the plant cell; and
(3) a third nucleic acid sequence encoding a termination region functional in said plant cell; and
(b) growing said plant cell to produce said TGF-β.

In a second aspect of the present invention there is provided a chimeric nucleic acid sequence comprising:

(1) a first nucleic acid sequence capable of regulating the transcription and/or translation in a plant cell of
(2) a second nucleic acid sequence, encoding a TGF-β, and adapted for expression in a plant cell; and
(3) a third nucleic acid sequence encoding a termination region functional in a plant cell

The methods and nucleic acids of the invention are particularly suitable for use in the expression of TGF-βs in a chloroplast of a plant cell. In particular, the chimeric nucleic acid may be one that is suitable for use in transformation of the chloroplast genome. Suitable chimeric nucleic acids may be suitable to be expressed in a plant chloroplast, and may preferably be adapted to be expressed in this manner. Preferred means by which nucleic acids (either the chimeric nucleic acid as a whole, or the first, second or third nucleic acids making up the chimeric nucleic acid) may be adapted for expression in the chloroplasts of plant cells are described throughout the specification.

The expression of proteins in chloroplasts, and in particular chloroplast transformation, provides many advantages over expression elsewhere in a plant cell. Compartmentalisation of expressed exogenous proteins in the chloroplast reduces their potential toxicity to the cell in which they are expressed. The chloroplast genome is present at high copy number, and may therefore be used to achieve high expression levels. Homologous recombination allows precise insertion of nucleic acids of interest, and continued stable expression of their products. Such expression may be observed in a wide range of plants. Finally, maternal inheritance in many crop plants means that the risk of unwanted transmission of transgenes in pollen is much reduced.

A “first nucleic acid sequence” of the type referred to in the first and second aspects of the invention, is one that is capable of regulating the transcription and/or translation in a plant cell of a second nucleic acid sequence (as defined elsewhere). A first nucleic acid sequence in accordance with the invention that is able to regulate the translation of a second nucleic acid sequence will preferably comprise a promoter site. Details of suitable promoter sites that may be incorporated in first nucleic acid sequences in accordance with the invention are considered elsewhere in the specification. A first nucleic acid sequence in accordance with the invention that is able to regulate the transcription of a second nucleic acid sequence will preferably comprise a ribosome binding site (RBS). Details of suitable RBSs that may be incorporated in first nucleic acid sequences in accordance with the invention are considered elsewhere in the specification. It will generally be preferred that a first nucleic acid sequence in accordance with the invention will be one that is capable of regulating both the transcription and translation of a second nucleic acid sequence. Accordingly a preferred first nucleic acid sequence may comprise both a suitable promoter and a suitable RBS. Preferred promoters and RBSs that may be used in such combined first nucleic acid sequences are described elsewhere in the specification. Preferred first nucleic acid sequences may be adapted for the regulation of transcription and/or translation in a chloroplast of a plant cell.

A “second nucleic acid sequence” in accordance with the present invention is a sequence that encodes a TGF-β to be expressed, and that is adapted for expression in a plant cell. The translation and/or transcription of the second nucleic acid sequence may be regulated by an appropriate first nucleic acid sequence, as described above. It will be appreciated that a suitable second nucleic acid sequence may encode any TGF-β that it is desired to express in a plant cell. A preferred, second nucleic acid may, for example, encode TGF-β1, TGF-β2, or TGF-β3, of which TGF-β3 may be more preferred. Second nucleic acids of the invention may be adapted for expression in a plant cell using one or more of various adaptation strategies. Examples of suitable adaptation strategies are described elsewhere in the specification. Preferred second nucleic acid sequences may be adapted for their expression in a chloroplast of a plant cell.

A “third nucleic acid sequence” in accordance with the present invention is a sequence that encodes a termination region that may be used to terminate translation of the second nucleic acid. The termination region will be one that is functional in a plant cell. Preferably a suitable termination region will be one that is functional in a chloroplast of a plant cell. Examples of suitable third nucleic acid sequences that may be used in accordance with the present invention (including sequences suitable for use in plant cells and chloroplasts) are considered elsewhere in the specification.

The first and/or second and/or third nucleic acid sequences may be preferably be genetically fused to one another, thereby producing a single chimeric nucleic acid molecule comprising the various nucleic acid sequences.

It is preferred that a chimeric nucleic acid sequence of the invention is a DNA sequence. Accordingly, it will be appreciated that preferred first and/or second and/or third nucleic acid sequences are DNA sequences.

In its broadest construction, the term “adapted for expression in a plant cell” may be taken to encompass any nucleic acid that may be expressed in a plant cell in order to achieve the requisite activity or expression. Various strategies may be employed to facilitate expression of chimeric nucleic acids in chloroplasts. Several such suitable strategies are discussed in further detail elsewhere in the specification, and these particular strategies may represent preferred means by which nucleic acids are to be adapted for expression in a plant cell.

Nucleic acids of the invention may be incorporated in suitable plasmids, such as chloroplast targeting plasmids. It may generally be preferred that nucleic acids that are to be expressed in chloroplasts be flanked by regions of plastid-targeting DNA that allow for insertion of the chimeric nucleic acid molecule in the chloroplast genome. Suitable plasmids represent preferred agents for use in the methods of the invention.

The TGF-β to be expressed may be any TGF-β derived from any animal, human or non-human, but preferably the TGF-β is a human TGF-β.

The methods or nucleic acids of the invention may be used to express any TGF-β (e.g. any of TGF-β1, TGF-β2, TGF-β3, TGF-β4 or TGF-β5). It is preferred that the TGF-β, is selected from the group consisting of TGF-β1, TGF-β2 and TGF-β3. It is even more preferred that the TGF-β be TGF-β3. It is particularly preferred that the TGF-β is human TGF-β3.

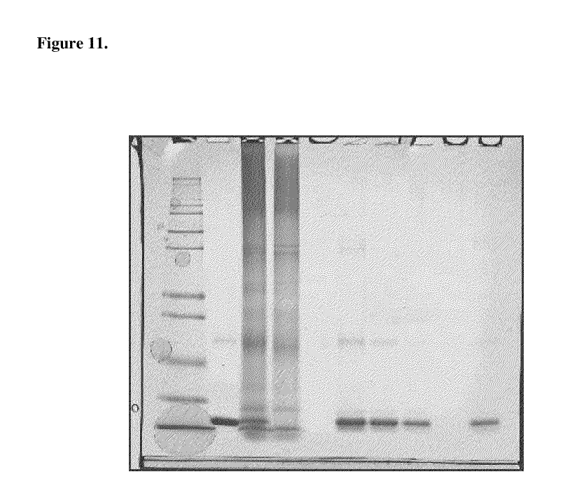

It will be appreciated that the TGF-β encoded by a nucleic acid sequence in accordance with the present invention will preferably comprise the active fragment of TGF-β. The TGF-β encoded may suitably comprise the active fragment alone (i.e. without association of the latency associated peptide). Suitable nucleic acids may encode all or part of the selected TGF-β active fragment. For reference, the amino acid sequences of the active fragments of TGF-β1, TGF-β2 and TGF-β3 are set out as Sequence ID Nos. 1 to 3 respectively in FIG. 11.

The TGF-β encoded by a nucleic acid sequence in accordance with the invention, or expressed in a method according to the invention, may comprise a TGF-β proprotein. Such proproteins may exhibit stability that makes them suitable for long-term storage or processing prior to commercial use. The inventors believe that that purification of the proprotein homodimer (75 kDa) may be easier than active region homodimer (24 kDa), due to protein stability. Encapsulation of the active protein may increase the protein half-life by 40-fold, and engineered cleavage sites released the protein at its therapeutic site of action. The proprotein may be cleaved in vitro after purification to provide the active region, for instance for use as a therapeutic agent.

A TGF-β encoded by a nucleic acid sequence in accordance with the invention, or expressed in a method according to the invention, may comprise a full length TGF-β, preferably a full length TGF-β having an amino acid sequence as encoded by any one of Sequence ID Nos. 6, 7 or 8.

A TGF-β encoded by a nucleic acid sequence in accordance with the present invention may comprise a variant form of TGF-β.

The methods and nucleic acids in accordance with the present invention may employ a promoter derived from a gene expressed in the chloroplast of plants. It may be preferred that a suitable promoter be derived from a photosynthetic gene.

A suitable promoter for use in the methods or nucleic acids of the present invention may be selected from the group consisting of plastid promoters comprised of promoters expressing photosynthesis-related genes, genetic system genes and any others which are recognised by the plastid encoded plastid (PEP) RNA polymerase or nucleus-encoded plastid (NEP) RNA polymerase, algal promoters, bacterial promoters or phage promoters such as the plastid psbA promoter, plastid 16S rrn promoter, Chlamydomonas psbA promoter, bacterial trc promoter and bacteriophage T7 promoter. Of this group, a 16srrn promoter represents a preferred promoter. A suitable promoter may be derived from Nicotiana tabacum, or preferably from Brassica napus. Indeed, the Brassica napus 16srrn promoter represents a particularly preferred promoter for use in the methods or nucleic acids of the invention.

A suitable ribosome binding site (RBS) for use in the methods or nucleic acids of the invention may be selected from the group consisting of any plastid RBS such as the rbcL RBS or psbA RBS, or bacterial or bacteriophage RBS such as the T7g10 RBS. Of this group, a T7g10 RBS may be a preferred RBS. Other suitable RBSs for use in the methods of the invention include those derived from Nicotiana tabacum, such as the Nicotiana tabacum psbA RBS.

A suitable terminator that may be used in the methods or nucleic acids of the invention may be selected from the group consisting of plastid terminators including the psbA terminator, rbcL terminator, a rps18 terminator (from ribosomal protein S18) and a psbC terminator or a bacterial terminator or bacteriophage terminator. A psbC terminator represents a favoured terminator from this group. Suitable terminators may be derived from Hordeum vulgare, or preferably from Brassica napus. A Brassica napus psbC terminator is a particularly preferred terminator for use in accordance with the present invention.

As set out above, chloroplast expression may be used to achieve expression of a TGF-β, in accordance with the invention in a wide variety of plants. The inventors believe that the methods and nucleic acids of the invention (and in particular those used in chloroplast expression) may be used in either monocotyledonous plants or dicotyledonous plants. A preferred example of a dicotyledonous plant that may be utilised in accordance with the methods and nucleic acids of the invention is tobacco. Generally, the inventors believe that the methods and nucleic acids of the invention may be used in connection with a wide range of plants, including land plants and algae. Suitable plants include, but are not limited to, cabbage, cauliflower, Chlorella, Chlamydomonas, barley, carrot, lettuce, moss, maize, oil seed rape, pepper, potato, rice, soybean, sunflower, tomato, wheat. Methods or nucleic acids in accordance with the present invention may make use of appropriate targeting sequences and promoters selected with reference to the selected plant in which they are to be expressed. For example, suitable nucleic acids or methods in accordance with the present invention may make use of targeting sequences and promoters that are suitable for use in algae or mosses.

As set out above, the inventors believe that the expression of TGF-βs in the chloroplast, and particularly such expression occurring as a result of transformation of the chloroplast genome, provides many advantages in the context of the present invention. The inventors have identified a number of strategies that may be used in producing a nucleic acid encoding a TGF-β, and which is adapted for expression in a chloroplast.

Expression of TGF-βs in accordance with the present invention may be achieved by the use of regulatory nucleic acid sequences (in “first nucleic acid sequences” as referred to elsewhere in the specification, which may comprise promoters and ribosome binding sites), that are suitable for chloroplast expression, and preferably may make use of first nucleic acid sequences which are preferential for, or even specific for, chloroplast expression.

In the same manner, the methods and nucleic acids of the invention may make use of termination regions (in “third nucleic acid sequences” as referred to elsewhere in the specification) that are suitable for expression in the chloroplast. Such third nucleic acid sequences may even more preferably be preferential for, or specific for, expression in a chloroplast.

In particular, the adaptation of nucleic acids for expression in plant cells may be undertaken with reference to sequences encoding the TGF-β to be expressed (“second nucleic acid sequences” as considered herein). The inventors have identified a number of means that may be used to adapt such second nucleic acid sequences for expression in a plant cell, and more particularly for expression in a chloroplast. The use of one or more of these means in the generation of suitable second nucleic acid sequences may be a preferred embodiment of any of the methods or nucleic acid sequences described in the present invention.

One preferred method by which such second nucleic acid sequences may be adapted for expression in a plant cell, or more particularly in a chloroplast, is the substitution of one or more of the codons found in the native DNA encoding the TGF-β to be expressed.

In a particularly preferred embodiment, it may be preferred to substitute one or more of the codons encoding the amino acid cysteine occurring in the native DNA. TGF-βs comprise a number of cysteine residues, and these residues are characteristic of the TGF-β proteins. However, cysteine is an amino acid that is found in lower amounts than other amino acids in chloroplast gene products, and significantly lower amounts in photosynthetic chloroplast gene products.

The inventors have found that DNA encoding a TGF-β may be adapted for expression in a chloroplast of a plant cell if one (or more) UGC codons present in native DNA encoding the TGF-β (e.g. the DNA of Sequence ID No. 4, in the case of the active fragment of TGF-β3) is substituted. The UGC codon encodes the amino acid cysteine, and a preferred substitution in such cases will generally be with the alternative cysteine-coding codon UGU. Preferably at least two of the UGC codons present in a native DNA sequence may be substituted, more preferably at least three of the UGC codons may be substituted, and most preferably four of the UGC codons may be substituted. The inventors believe that five, or even six, of the UGC codons present in the native DNA may be substituted and still allow production of a desired TGF-β, however it is preferred that at least one, and more preferably two, UGC codons are retained in a suitable nucleic acid.

The inventors have identified a number of other codons that may be the subject of alternative, or further, substitutions.

For example, the leucine-encoding codon CUG may beneficially be subject to substitution in the production of nucleic acids adapted for expression in plant cells (and in particular in chloroplasts of plant cells). It may be preferred that at least one CUG codon is substituted to produce a second nucleic acid sequence suitable for use in the methods or nucleic acid sequences of the invention. For example, it may be preferred that all CUG codons present in a native DNA encoding the TGF-β to be expressed are substituted. For example, in the case of a native DNA encoding human TGF-β3, it may be preferred to substitute all seven CUG codons present. A preferred substitute codon to be used may be the alternative leucine-encoding codon UUA.

Additionally or alternatively, the valine-encoding codon GUG may beneficially be subject to substitution when producing nucleic acids adapted for expression in plant cells (and in particular in chloroplasts of plant cells). It may be preferred that at least one GUG codon is substituted to produce a second nucleic acid sequence suitable for use in the methods or nucleic acid sequences of the invention. For example, it may be preferred that all GUG codons present in a native DNA encoding the TGF-β, to be expressed are substituted. For example, in the case of a native DNA encoding human TGF-β3, it may be preferred to substitute all six GUG codons that would otherwise be present. Preferred substitute codons to be used may be the alternative valine-encoding codons GUU or GUA.

As an alternative, or in addition, the proline-encoding codon CCC may beneficially be substituted in the production of nucleic acids adapted for expression in plant cells (and in particular in chloroplasts of plant cells). It may be preferred that at least one CCC codon is substituted to produce a second nucleic acid sequence suitable for use in the methods or nucleic acid sequences of the invention. For example, it may be preferred that all CCC codons that are otherwise present in a native DNA encoding the TGF-β to be expressed are substituted. For example, in the case of a native DNA encoding human TGF-β3, it may be preferred that all four of the CCC codons that would otherwise be present are substituted. A preferred substitute codon to be used may be the alternative proline-encoding codon CCU.

By way of further alternative or addition, the tyrosine-encoding codon UAC may beneficially be substituted to produce nucleic acids adapted for expression in plant cells (and in particular in chloroplasts of plant cells). It may be preferred that at least one UAC codon is substituted to produce a second nucleic acid sequence suitable for use in the methods or nucleic acid sequences of the invention. For example, it may be preferred that at least one, two, three or four UAC codons present in a native DNA encoding the TGF-β3 to be expressed are substituted. For example, in the case of a native DNA encoding human TGF-β3, it may be particularly preferred that five of the six UAC codons that would otherwise be present are substituted. A preferred substitute codon to be used may be the alternative tyrosine-encoding codon UAU.

In a still further alternative or addition to the adaptations described above, it may be preferred that the asparagine-encoding codon AAC be substituted in the production of nucleic acids adapted for expression in plant cells (and in particular in chloroplasts of plant cells). It may be preferred that at least one AAC codon is substituted to produce a second nucleic acid sequence suitable for use in the methods or nucleic acid sequences of the invention. For example, it may be preferred that at least one, two, three or four AAC codons present in a native DNA encoding the TGF-β3 to be expressed are substituted. For example, in the case of a native DNA encoding human TGF-β3, it may be particularly preferred that five of the six AAC codons that would otherwise be present are substituted. A preferred substitute codon to be used may be the alternative asparagine-encoding codon AAU.

Another adaptation that may be used in addition or alternative to those described above in the production of nucleic acids adapted for expression in plant cells (and in particular in chloroplasts of plant cells), is the substitution of the aspartic acid-encoding codon GAC. It may be preferred that at least one GAC codon is substituted to produce a second nucleic acid sequence suitable for use in the methods or nucleic acid sequences of the invention. For example, it may be preferred that all GAC codons present in a native DNA encoding the TGF-β to be expressed are substituted. For example, in the case of a native DNA encoding human TGF-β3, it may be particularly preferred all four of the GAC codons that would otherwise be present are substituted. A preferred substitute codon to be used may be the alternative aspartic acid-encoding codon GAU.

For the purposes of the present disclosure native DNA should be considered to be the naturally occurring DNA encoding a TGF-β to be expressed or encoded in accordance with the invention. For example, in the case of human TGF-β1 (the amino acid sequence of the active fragment of which is set out in Sequence ID No. 1), the native DNA will be the naturally occurring human genomic DNA encoding this protein (the full length DNA sequence of which is shown in Sequence ID No. 6). In the case of human TGF-β2 (the amino acid sequence of the active fragment of which is set out in Sequence ID No. 2), the native DNA will be the naturally occurring human genomic DNA encoding this protein (the full length DNA sequence of which is shown in Sequence ID No. 7). In the preferred case of human TGF-β3 (the amino acid sequence of the active fragment of which is set out in Sequence ID No. 3), the native DNA will be the naturally occurring human genomic DNA encoding this protein (for instance, the DNA encoding the active fragment, as set out in Sequence ID No. 4, or the full length DNA sequence shown in Sequence ID No. 8).

An example of a particularly preferred nucleic acid sequence encoding a TGF-β (in this case the active fragment of TGF-β3) and adapted for expression in a plant cell, and more particularly in a chloroplast, is shown in Sequence ID No. 5. Indeed, so preferred is this nucleic acid sequence that in a further aspect of the invention there is provided a nucleic acid sequence comprising the nucleic acid sequence set out in Sequence ID No. 5. The nucleic acid sequence set out in Sequence ID No. 5 represents both a preferred second nucleic acid sequence for use in the methods of the invention, and also a preferred second nucleic acid sequence for use in the nucleic acids of the invention.

The inventors believe that a nucleic acid sequence sharing at least 1.75% codon identity with the sequence set out in Sequence ID No. 5 may be utilised in the methods and nucleic acids of the invention, on the proviso that such a nucleic acid sequence still encodes a TGF-β to be expressed. More preferably a suitable nucleic acid may share at least 22% codon identity with Sequence ID No. 5, even more preferably at least 50% codon identity, still more preferably at least 75% codon identity, and most preferably at least 99.1% codon identity.

It will be appreciated that nucleic acid sequences described in the preceding paragraphs, such as the nucleic acid sequence of Sequence ID No. 5 (or sequences sharing the specified degrees of identity, such as at least 22% codon identity with Sequence ID No. 5), may comprise suitable “second nucleic acid sequences” for use in accordance with any or all of the methods or nucleic acids of the invention.

The inventors have found that modifications of the type described above are very effective in increasing the total amount of a TGF-β that can be expressed in a plant cell (including expression in the chloroplast). For example, as explained further in the Experimental Results section below, plants transformed with a nucleic acid comprising the native DNA encoding TGF-β3 may give rise to a yield of TGF-β3 that is approximately 1% of total protein. By way of contrast, use of nucleic acid sequences adapted for expression in a plant cell, such as the nucleic acid sequence of Sequence ID No. 5, are able to produce yields of TGF-β3 ten times higher than those produced using the native sequence (giving rise to a yield of TGF-β3 that is approximately 10% of total protein). The inventors have found that the use of selected second nucleic acid sequences of this sort, such as Sequence ID No. 5 are able to significantly increase TGF-β yield compared to native sequences, even when the same first and third nucleic acid sequences are used in common.

It will be appreciated that these increases in TGF-β yield represent a remarkable and surprising improvement over that which may otherwise be achieved without utilising methods and nucleic acids of the invention. The amount of TGF-β produced utilising the methods and nucleic acids of the invention allow economically advantageous production of TGF-βs (such as TGF-β3) in plants in a manner that was not previously possible.

The inventors have further identified a number of new techniques and conditions that may optionally be used advantageously in the methods of the invention. These provide notable benefits in terms of recovery of TGF-β expressed in accordance with the invention, and/or the folding or re-folding of such TGF-β to produce active TGF-β. The novel methods developed also include procedures suitable for use in the capture of re-folded TGF-β that has been expressed in a method according to the invention.

Recombinant proteins expressed in plants are typically expressed as soluble proteins. This is generally considered to be due to the relatively low levels of protein expression that may be achieved using the methods described in the prior art. The soluble proteins produced tend to comprise a mixture of biologically active and biologically inactive forms, with inactive forms representing the greater proportion of the total.

The high levels of expression achieved using the methods and nucleic acids of the invention were found to produce a high yield of recombinant TGF-β protein, but to give rise to insoluble aggregations of these proteins, with no detectable protein expressed in a form correctly folded to produce biological activity. Without wishing to be bound by any hypothesis, the inventors believe that these aggregates arise due to the high concentration of recombinant protein established within the plant cells (and particularly the chloroplasts), and as a result of the hydrophobicity of the TGF-β proteins expressed. The production of insoluble aggregates of TGF-β in this manner has advantages (in that it is easier to separate the insoluble recombinant protein from soluble plant cell components that may otherwise constitute contaminants), and this insoluble form of the TGF-β represents a useful product in itself (since it may subsequently be solubilised and folded to its active form using prior art techniques). However, in order to produce correctly folded biologically active forms of TGF-β with improved purity and yield, the inventors developed new techniques particularly suited to the solubilisation and folding/re-folding of TGF-β expressed using the methods and nucleic acids of the invention.

The inventors have found that an advantageous step in the purification of TGF-β expressed using the methods or nucleic acids of the invention involves the lysis of chloroplast extracts (in which TGF-β has been expressed within the chloroplasts) and homogenisation and sonication of the resulting mixture to aid dissolution of the TGF-β. Lysis may be achieved using a buffer comprising 10 mM HEPES, 5 mM EDTA, 2% weight/weight Triton X-100, 0.1M DTT at pH 8.0.

TGF-β expressed using the methods or nucleic acids of the invention may advantageously be “washed” to remove contaminants, such as chlorophyll, or other plant proteins. A suitable wash buffer may comprise 0.05M Tris base and 0.01M EDTA at pH 8.0. Washing may readily be carried out by a series of centrifugation and re-suspension steps preferably two or more cycles of centrifugation and re-suspension in a wash buffer). Centrifugation may be carried out at 8000×g for 30 minutes.

The TGF-β product obtained after such washing may then be solubilised, preferably using a solvent that dissolves the recombinant TGF-β, but not plant proteins or carbohydrates (such as starch). The inventors have found that a suitable buffer having this activity may comprise urea, and a preferred example of such a buffer comprises 0.05M Tris base, 0.1M DTT, 6M Urea at pH 8.0. Such solubilisation may be achieved at room temperature (preferably with stirring to aid solubility) and may be aided by adjusting the pH of the solubilising solution to around 9.5. This use of a solvent capable of preferentially solubilising recombinant TGF-β, but not plant cell components (such as plant proteins or carbohydrates) has not been suggested in the prior art and, due to the notable advantages that it confers, represents a preferred step that may be utilised in the methods of the invention.

When TGF-β, expressed using the methods or nucleic acids of the invention, has been solubilised (for instance in the manner outlined above) it may then be concentrated using a diafiltration technique. A suitable technique may utilise a 5 kDa TFF (tangential flow filtration) membrane and a diafiltration buffer comprising 0.05M Tris base, 0.01 M DTT, 3M Urea at pH 9.5. Such diafiltration may be used to concentrate the solution by about 15 fold.

A TGF-β produced in accordance with any embodiment of the methods of the invention may be folded or re-folded using a technique in which folding occurs in the presence of CHES (2-(cyclohexylamino)ethanesulfonic acid), or a functional analogue thereof, such that active TGF-β is produced. Folding or re-folding of TGF-β in this manner is particularly advantageous, and methods incorporating this further step represent preferred embodiments of the invention. Preferably the CHES may be used at a concentration of about 100 nM to 1.0 M, more preferably at a concentration of about 0.7M. Optional steps involving use of CHES in folding of TGF-βs expressed using the methods or nucleic acids of the invention may utilise CHES (or a functional analogue thereof) in combination with a low molecular weight sulfhydryl/disulfide redox system. Further details of folding or re-folding methods utilising CHES that may advantageously be used in the methods of the present invention are include in International Patent Application PCT/GB2007/000814, and the contents of this document are incorporated herein by reference, particularly insofar as they relate to methods for folding TGF-βs to produce biologically active molecules.

TGF-β expressed in accordance with the methods of the invention may be captured by hydrophobic interaction chromatography. By way of example, Butyl-Sepharose 4 Fast Flow separation medium may be used to implement such capture. A solution comprising the TGF-(preferably re-folded to an active form in the manner described above) may be added to the Butyl-Sepharose 4 Fast Flow column equilibrated with wash buffer and equilibration buffer. A suitable equilibration buffer may comprise 0.02M Sodium Acetate, 1 M Ammonium Sulphate, 10% volume for volume Acetic Acid, at pH 3.3. The column may be washed as appropriate prior to elution of bound TGF-β. Elution may utilise a suitable elution buffer, such as one comprising 0.02 M Sodium Acetate, 10% volume for volume Acetic Acid, 30% volume for volume Ethanol at pH 3.3.

The TGF-β may be further purified by cation exchange chromatography. By way of example, SP-Sepharose medium may be used to further purify the TGF-β dimer from TGF-β, monomer and plant related impurities. To ensure the binding of the TGF-β dimer to the cation exchange chromatography media, the conductivity of eluate from capture purification step (preferably from Butyl-Sepharose eluate described above) may need to be lowered and this is best achieved by diluting the eluate in a suitable buffer (for example a buffer containing 2.72 g/L sodium acetate trihydrate, 100 mL/L glacial acetic acid, 300 mL/L ethyl alcohol at pH 3.9-4.1). The conditioned load is then added to the SP-Sepharose column and equilibrated with a suitable buffer. The buffer may comprise 2.72 g/L sodium acetate trihydrate, 100 mL/L glacial acetic acid, 300 mL/L ethyl alcohol, 2.92 g/L sodium chloride at pH 3.9-4.1. The column may be washed as appropriate prior to elution of bound TGF-β. Elution of the TGF-β from the column can be achieved by changing the pH or by raising conductivity of the mobile phase. A suitable elution buffer, by way of an example would consist of 2.72 g/L sodium acetate trihydrate, 100 mL/L glacial acetic acid, 300 mL/L ethyl alcohol, 29.22 g/L sodium chloride at pH 3.9-4.1. Fractions of the SP Sepharose eluate containing TGF-β dimer should be pooled according to purity. Since residual salt can cause the aggregation of TGF-β proteins, the SP-Sepharose eluate should be buffer exchanged into a suitable final formulation an example buffer would compromise 1.2 mL/L acetic acid, 200 mL/L ethyl alcohol at pH 4.0±0.1.

The optional steps set out above, when employed individually or in combination in the methods of the invention confer marked advantages over prior art techniques that have been suggested for the purification of recombinant human proteins from plants, or for the purification of TGF-βs in general. Accordingly the skilled person will recognise that one or more (and preferably all) of these optional steps may be advantageously incorporated in the methods of the invention. In particular, the use of these new methods for purification of recombinant proteins allow highly purified TGF-βs, such as TGF-β3 to be produced without the need for salt precipitation and chromatography (techniques that are suggested by the prior art, but may lead to undesirable aggregation of proteins purified in this manner, due to the presence of residual salt).

The skilled person will readily appreciate that nucleic acids of the invention may be introduced into a plant cell (as required by the methods of the invention) through any suitable route. A range of techniques suitable for the introduction of nucleic acids in this manner are known to those skilled in the art, including, but not limited to, ballistic transfection. A suitable experimental protocol is described further in the Experimental Results section.

Nucleic acids in accordance with the invention may be further incorporated in suitable expression cassettes, or vectors. Examples of such expression cassettes or vectors will be well known to those skilled in the art of plant expression of proteins. Suitable examples of expression cassettes incorporating chimeric nucleic acid sequences in accordance with the present invention are set out in the Experimental Results section.

It may be preferred that chimeric nucleic acids of the invention (and suitable for use in the methods of the invention) further comprise nucleic acid sequences for the expression of products that may aid in the identification of plant cells into which the chimeric nucleic acid sequences have been successfully incorporated. Examples of suitable further nucleic acid sequences that may be used in this manner will be apparent to those skilled in the art, and include nucleic acids giving rise to products that confer resistance to substances that may be used for selection (such as antibiotics) or markers that give rise to a detectable product that may be used as the basis for selection (such as a chromogenic enzyme product).

In a further aspect the present invention provides a plant transformed with a nucleic acid according to the second aspect of the invention (and any embodiment thereof described in this specification).

In a further aspect the present invention provides a plant seed comprising a nucleic acid according to the second aspect of the invention (and any embodiment thereof described in this specification).

In addition to the methods and nucleic acids described elsewhere in the specification, the present invention also provides a TGF-β expressed by a method in accordance with the invention. The skilled person will appreciate that there are a number of distinguishing features by which the plant origins of such a TGF-β may be recognised. For example, in the case of a TGF-β proprotein the glycosylation that would be found in TGF-βs expressed by animal cells, or those expressed as a result of the nuclear transformation of plant cells, will be missing from proproteins expressed in the chloroplast. This may be used in the identification of proteins or proproteins produced in accordance with the invention.

The skilled person will appreciate that the methods and nucleic acids described in the present specification may be adapted, particularly by adaptation of the second nucleic acid sequences, for use in the expression of TGF-β superfamily members other than TGF-β isoforms themselves. Accordingly further aspects of the invention provide methods and nucleic acids in which the second nucleic acid sequence encodes a TGF-β superfamily member other than a TGF-β.

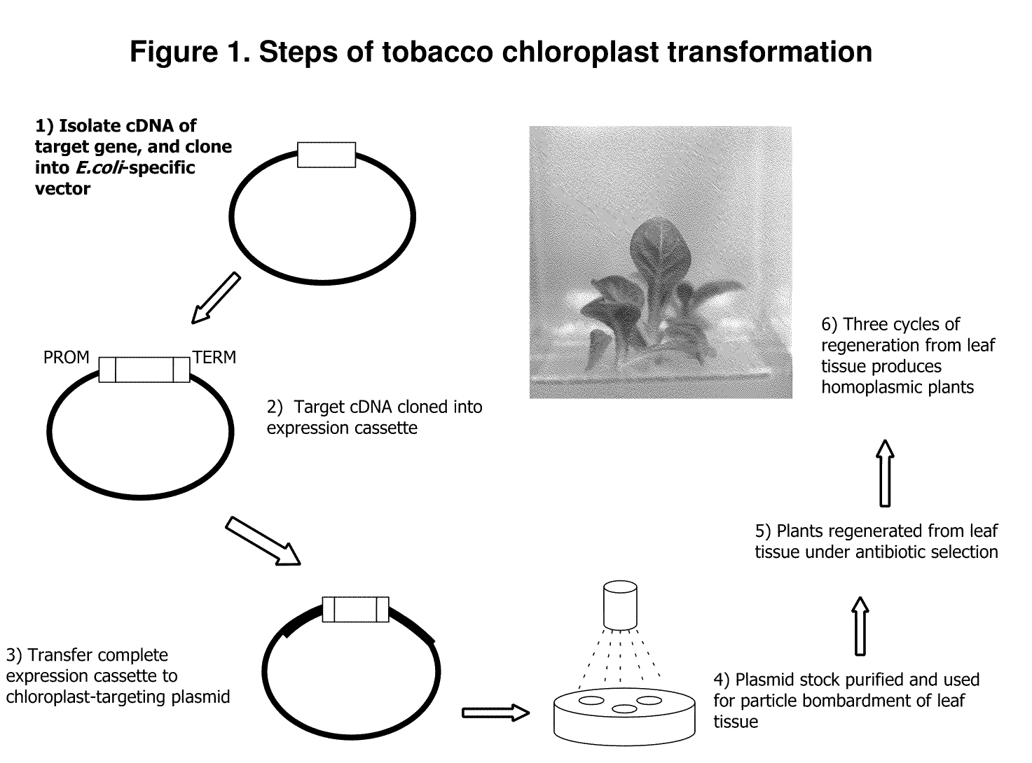

The invention will now be further described with reference to the following Experimental Results and accompanying FIGS. 1 to 12 in which:

FIG. 1 schematically shows the steps involved in tobacco chloroplast transformation to practice a method in accordance with the present invention. At 1, cDNA of the target TGF-β gene is isolated and cloned into an E. coli specific vector; at 2, the target cDNA is cloned into an expression cassette; at 3, the complete expression cassette is transferred to a chloroplast-targeting plasmid; at 4, the plasmid stock is purified and used for particle bombardment of leaf tissue; at 5, plants are regenerated from the leaf tissue under antibiotic selection conditions; and at 6, three cycles of regeneration from leaf tissue produces homoplastic plants.

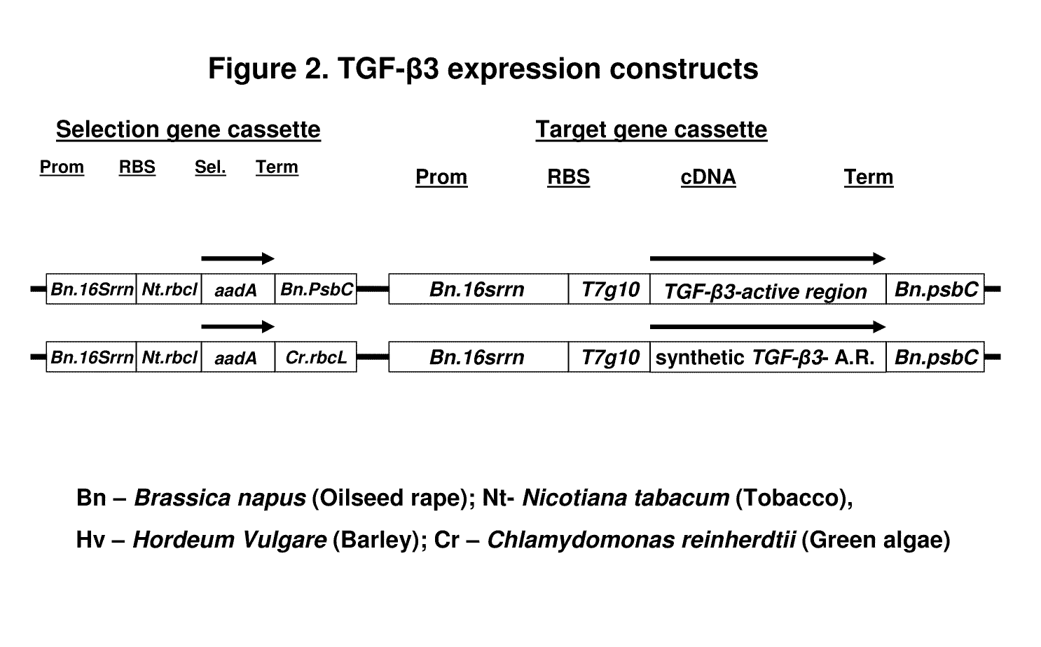

FIG. 2 illustrates, in schematic form, TGF-β3 expression constructs suitable for use in accordance with the present invention.

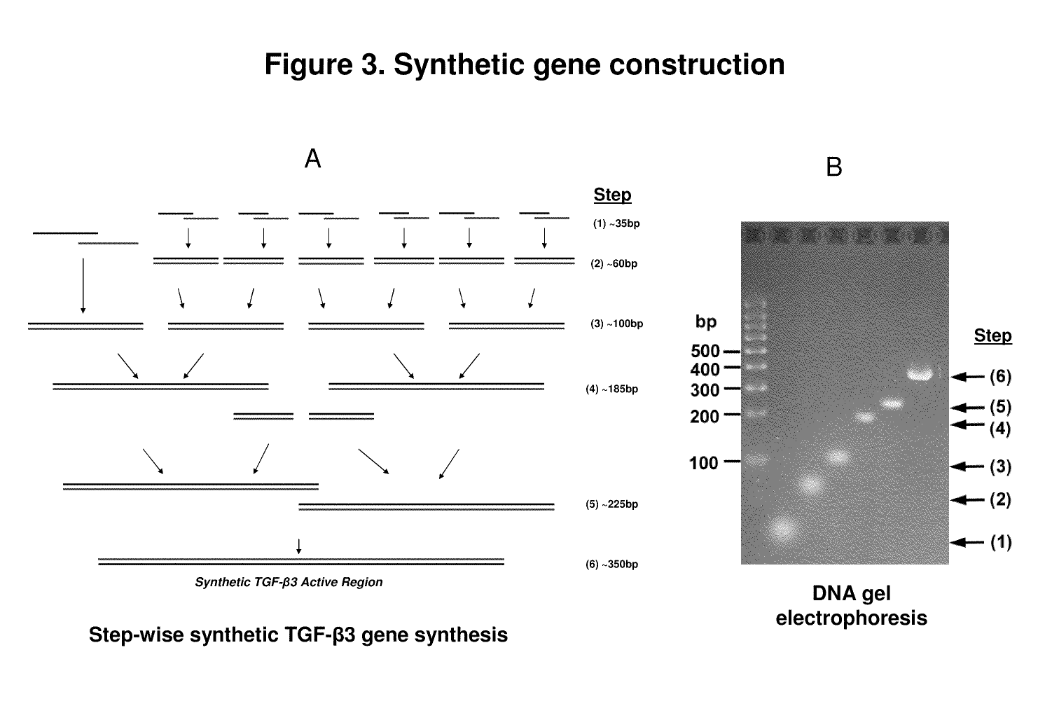

FIG. 3 illustrates synthetic gene construction to produce nucleic acids for use in the invention. In the left hand side of the Figure nucleic acid fragments are combined in a step-wise fashion to produce a synthetic TGF-β3 gene. The right hand side of the Figure shows DNA gel electrophoresis visualising the size of the different products yielded by the steps shown in the left hand panel.

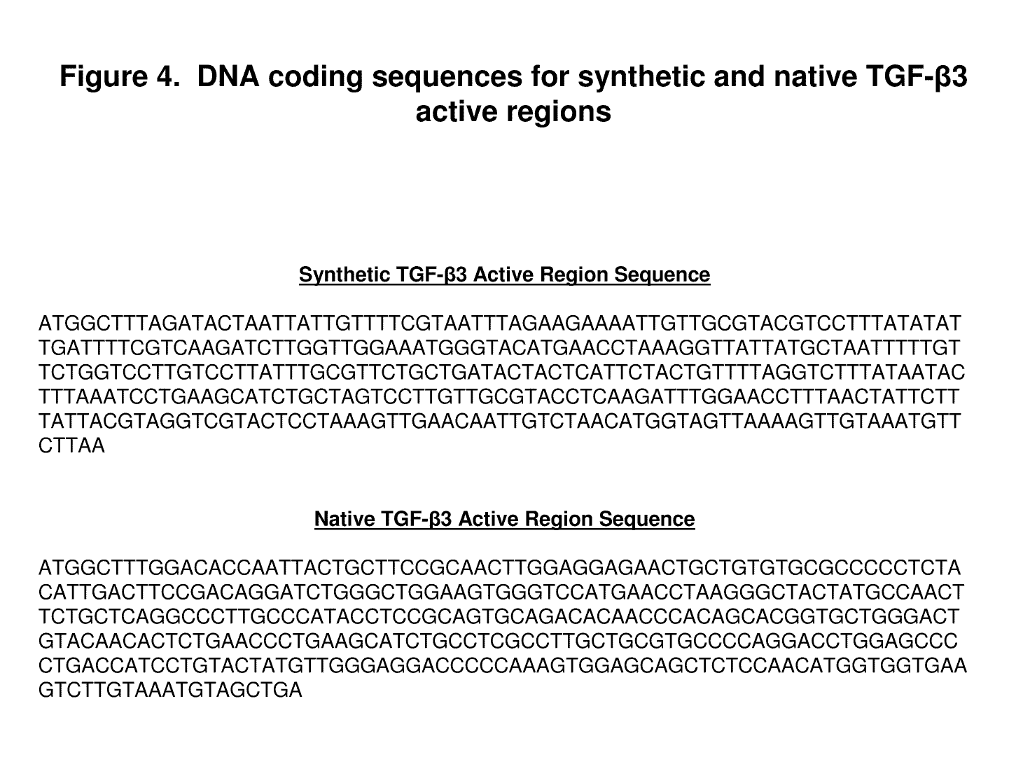

FIG. 4 compares the coding sequences of DNA from synthetic (upper sequence) and native (lower sequence) TGF-β3 active regions.

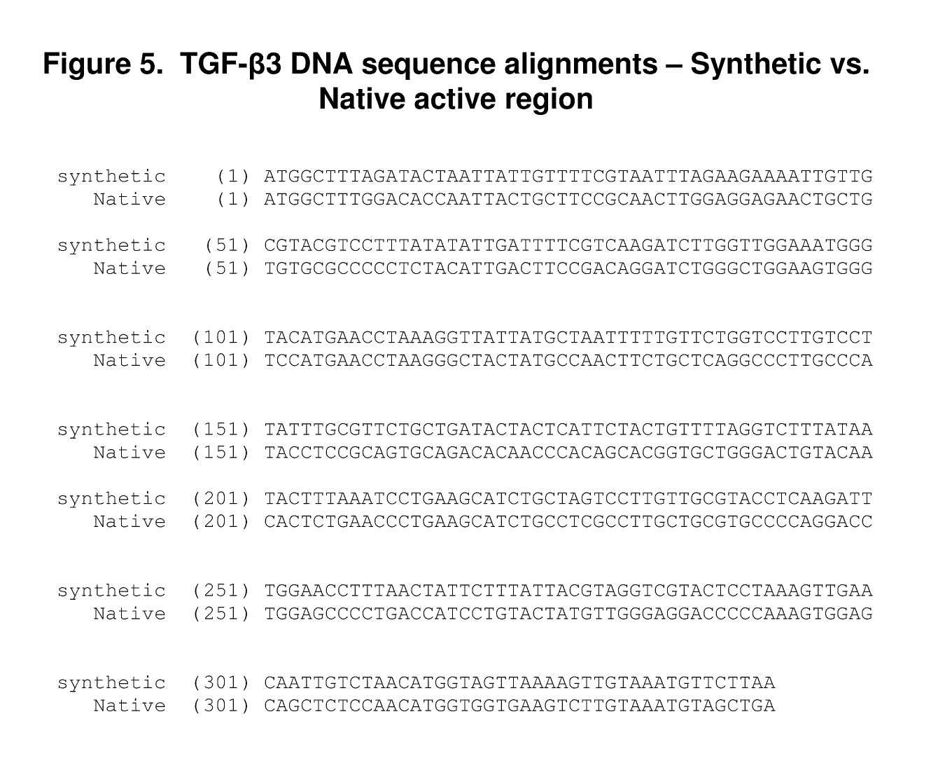

FIG. 5 shows alignments of the synthetic and native DNA sequences set out in FIG. 4.

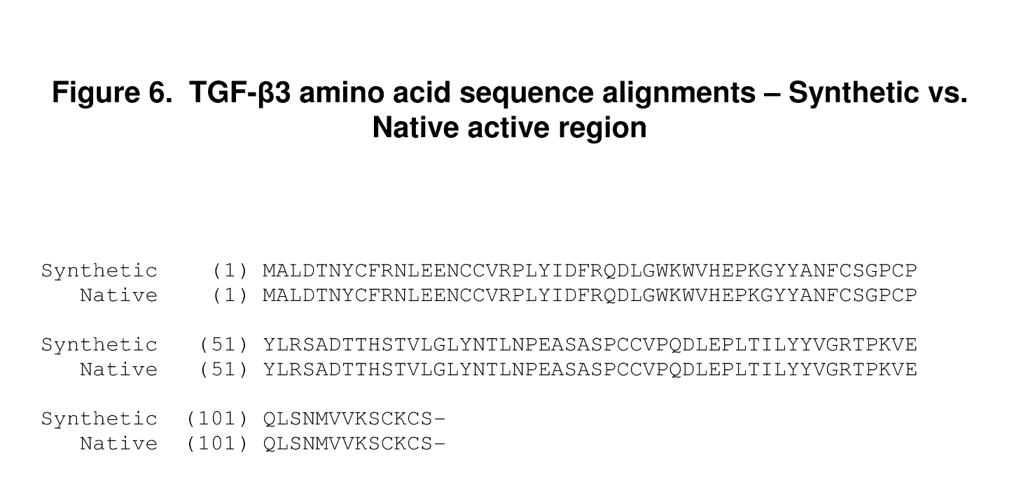

FIG. 6 shows alignments of the amino acid sequences of TGF-β3 encoded by the synthetic and native DNA sequences set out in FIGS. 4 and 5.

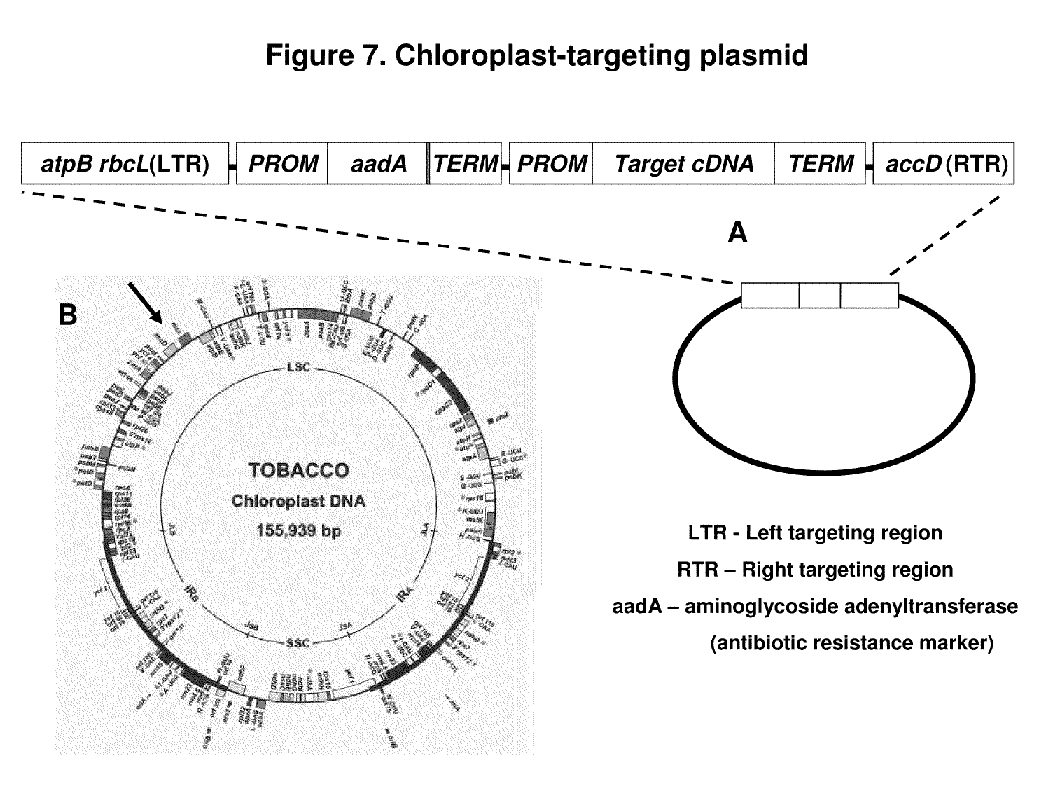

FIG. 7 schematically illustrates a chloroplast-targeting plasmid suitable for use in the present invention. “LTR” indicates the left targeting region and “RTR” indicates the right targeting region. “aadA” indicates aminoglycoside adenyltransferase, an antibiotic resistance marker that may be used.

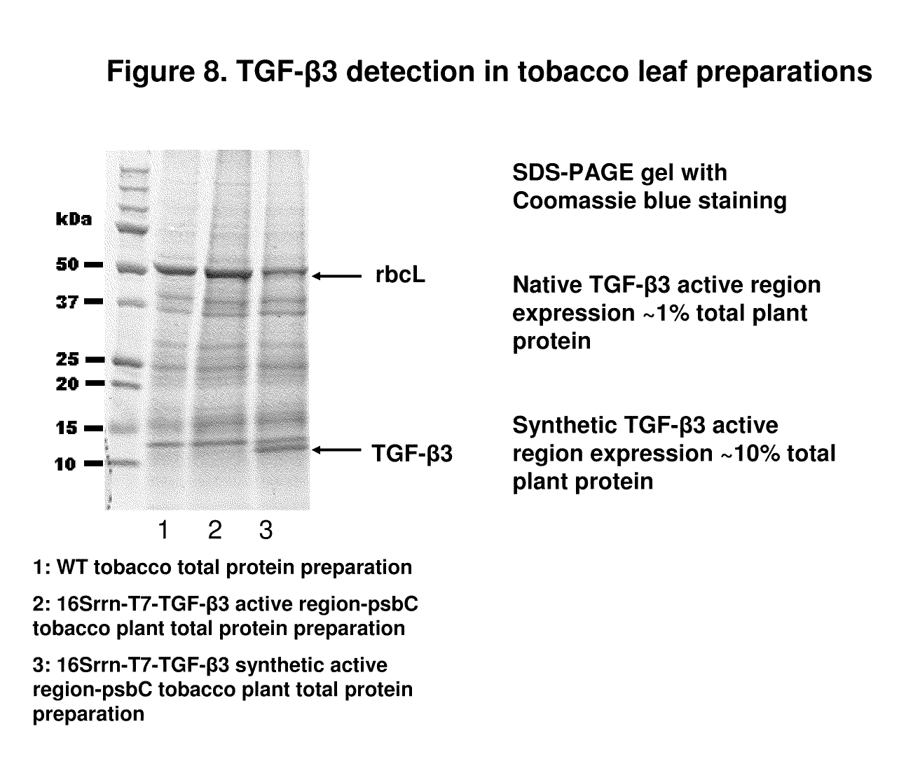

FIG. 8 illustrates detection of TGF-β3 produced in tobacco leaf preparations. The Figure shows an SDS-PAGE gel in which protein has been stained using Coomassie Blue. Yield is compared between total protein preparations derived from wild type tobacco plants (lane 1 of the gel), from 16Srrn-T7-TGF-β3 active region-psbC tobacco plants (i.e. plants in which the sequence of the nucleic acid encoding the TGF-β has not been adapted for expression in the plant cell—results shown in lane 2 of the gel), and from 16Srrn-T7-TGF-β3 synthetic active region-psbC tobacco plants (in which the sequence of the nucleic acid encoding the TGF-β has been adapted for expression in the plant cell—results shown in lane 3). Analysis of the results indicates that in this example TGF-β3 represents approximately 1% of the total protein in plants containing the native non-adapted sequence, and approximately 10% of the total protein in plants containing the synthetic adapted sequence.

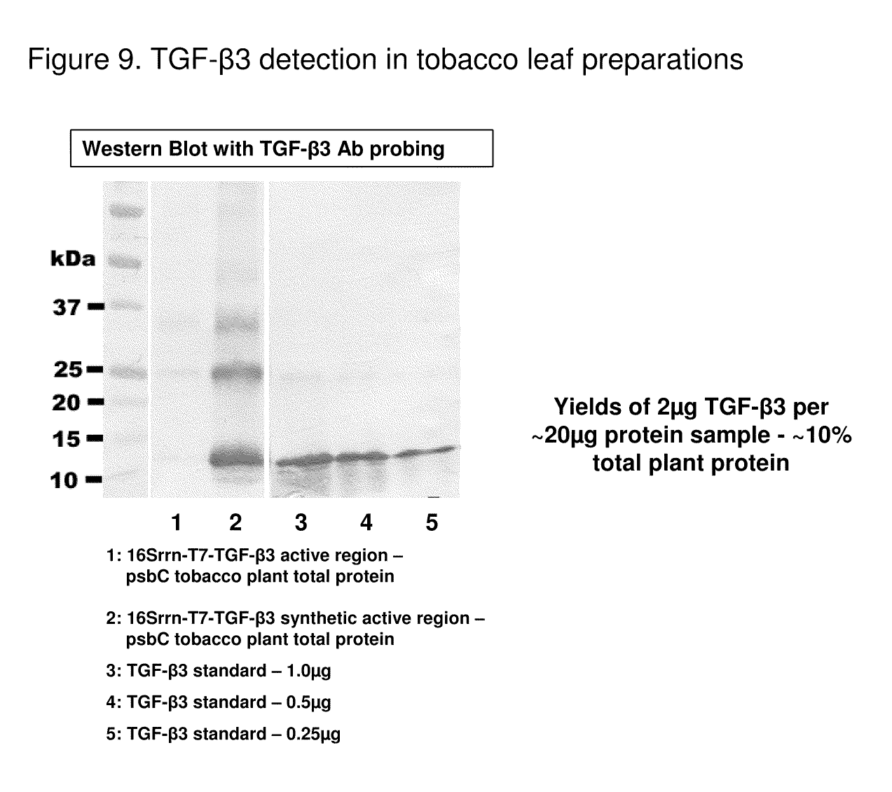

FIG. 9 also illustrates detection of TGF-β3 produced in tobacco leaf preparations, but in this case the Figure shows a Western blot (immunoblot) in which TGF-β3 has been labelled using an anti-TGF-β3 antibody. Lanes 1 and 2 compare yield in total protein preparations derived from 16Srrn-T7-TGF-β3 active region-psbC tobacco plants (shown in lane 1), and from 16 Srrn-T7-TGF-β3 synthetic active region-psbC tobacco plants (shown in lane 2). These are compared with TGF-β3 “standards” in lanes 3, 4 and 5 (1.0 μkg, 0.05 μg and 0.25 μg respectively). Analysis of the results indicates that in this example a 20 μg protein sample from plants containing the synthetic adapted sequence contained approximately 2 μg of TGF-β3 (i.e. approximately 10% of the total protein content).

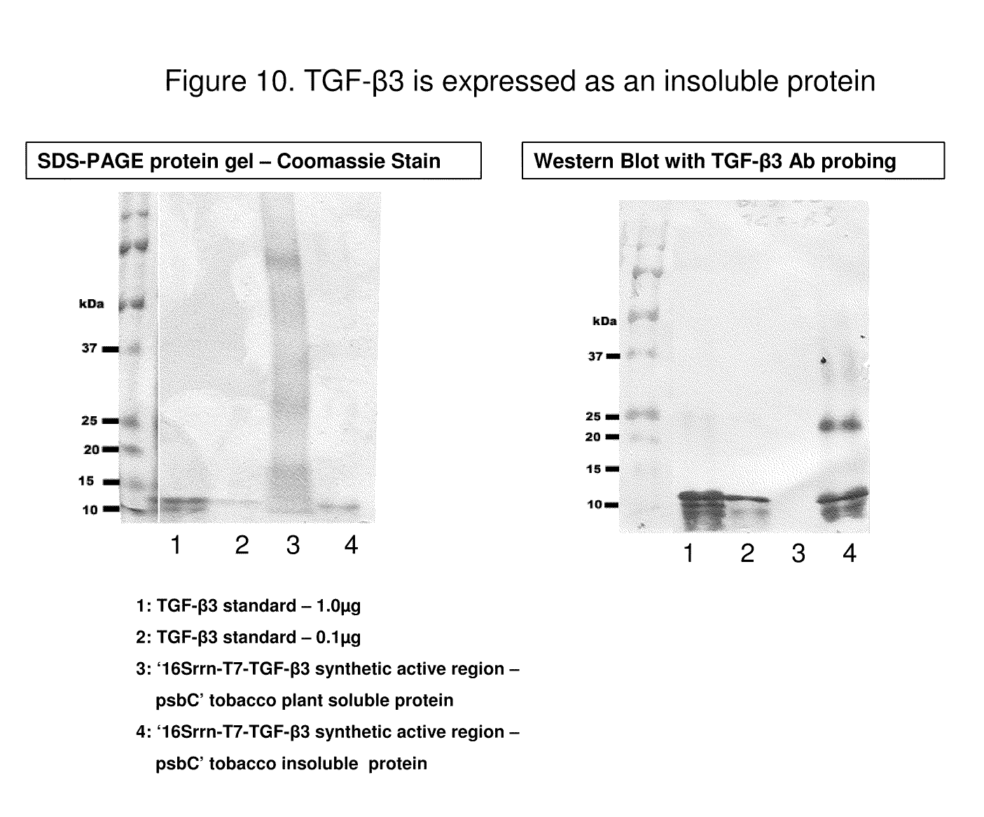

FIG. 10 illustrates that TGF-3 expressed by the methods described in the experimental results has the form of an insoluble protein. The left hand side of the Figure shows an SDS-PAGE gel in which protein has been stained using Coomassie Blue, whilst the right hand side shows a Western blot in which TGF-β3 has been labelled using an anti-TGF-β3 antibody. In both cases, lanes 1 and 2 are TGF-β3 “standards” (11.0 mg and 0.1 mg respectively), whereas lane 3 shows soluble protein collected from plants 16Srrn-T7-TGF-β3 synthetic active region-psbC tobacco plants and lane 4 shows insoluble protein collected from 16Srrn-T7-TGF-β3 synthetic active region-psbC tobacco plants.

FIG. 11 shows results obtained using a Biorad RC/DC assay to investigate recovery of material expressed by plants containing nucleic acids adapted for expression in plant cells.

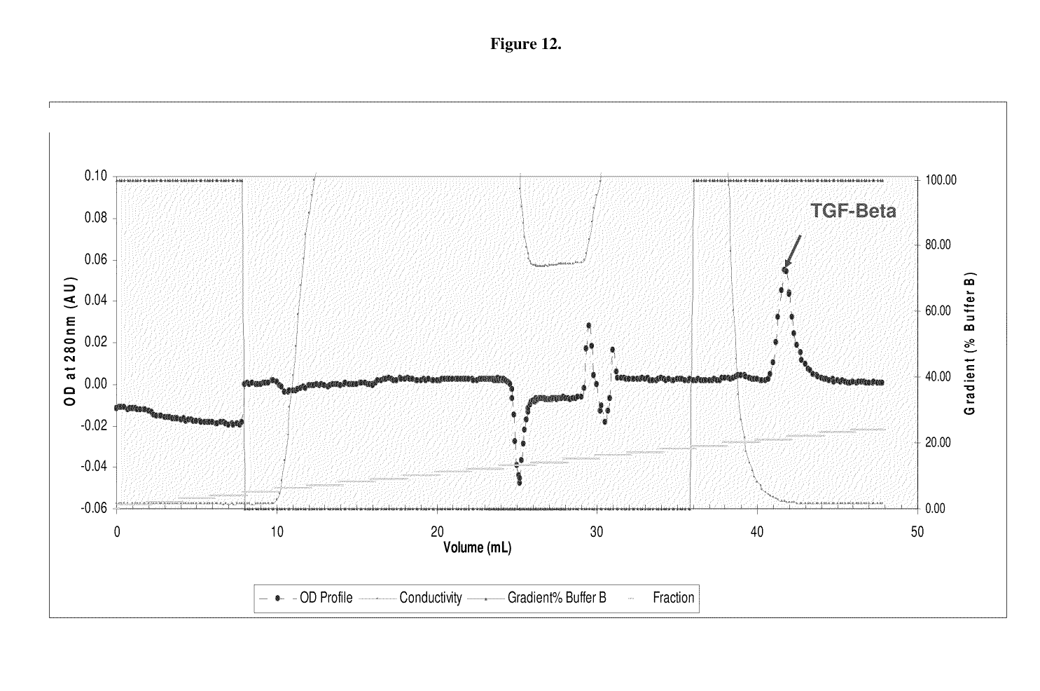

FIG. 12 shows a Butyl-Sepharose chromatogram illustrating yield of TGF-β3 from step elutions after Butyl-Sepharose capture.

Certain amino acid and nucleic acid sequences relied upon in the present disclosure are also set out in the Sequence Information section that follows the Experimental Results. As noted above, relevant sequences are also set out among the Figures.

### EXPERIMENTAL RESULTS

### 1. Introduction

The following describes an experimental protocol used to allow the expression of transforming growth factor beta 3 (TGF-β3) protein from tobacco (Nicotiana tabacum) plants, through genetic modification of the plants' chloroplast genomes.

An overview of the steps required to produce a transplastomic (plastid-modified genome) plant is shown in FIG. 1.

### 2. Results

### 2.1 Design of Expression Cassette Constructs

A number of expression cassettes were designed that contained DNA coding regions under the control of plastid-specific high-expression regulatory regions (see FIG. 2).

Regulatory regions from different species are often used for gene expression. These elements are similar enough to allow normal function in the non-native species, but differ in base sequence sufficiently to avoid homologous recombination into a non-target part of the plastome.

The expression cassettes shown in FIG. 2 contained the Brassica napus 16Srrn promoter and B. napus psbC 3′ terminator region, both plastid-specific. The RBS from the T7 bacteriophage gene 10 has also been incorporated into this expression cassette. The TGF-β3 active region coding region was integrated into this cassette. A synthetic TGF-β3 active region gene designed for optimal expression in the N. tabacum chloroplast (i.e. a second nucleic acid sequence in accordance with the present invention) was also synthesised and integrated into this expression cassette.

The 16Srrn promoter was selected since it can give rise to strong gene expression. The bacteriophage T7 gene 10 leader sequence is a ribosome binding site which has been used extensively in bacteria for high levels of translation, and has also been used in plastid expression successfully

All constructs also contained a marker gene aminoglycoside adenyltransferase (aadA) under control of plastid-specific regulatory regions. The aadA gene confers resistance to the antibiotics spectinomycin and streptomycin.

### 2.2 Construction of a Synthetic TGF-β3 Active Region Gene

A synthetic TGF-β3 active region gene was designed that was optimised for N. tabacum chloroplast gene expression. The gene was synthesised from single stranded oligonucleotides joined together in a step-wise method (see FIG. 3).

The first primer pair could not form a primer dimer, either due to internal hairpin formation or primer integrity, so a larger pair of primers were ordered at a higher cost to allow construction to continue quickly. At the joining of the two 185 bp primer “octomers” visualised in step 4, a final 350 bp product could not be achieved. It was thought this was a result of the 3′ single strand overlaps being too short in comparison to the total DNA strand lengths. Additional primer “dimers” already created in step 2 were joined onto the 180 bp constructs to create 225 bp DNA constructs with a large overlap. This method successfully overcame the problem and the final 350 bp synthetic TGF-β3 gene was amplified by PCR.

The synthetic sequence showed 70% base identity to the native DNA sequence, with a GC-content reduced from 56% to 33% in the optimised sequence. The DNA coding sequences of the synthetic TGF-β3 active region and native TGF-β3 active region are shown in FIG. 4. A DNA alignment of the synthetic and native sequences is shown in FIG. 5. The translated amino acid sequences for the synthetic and native sequences are identical and shown in FIG. 6.

### 2.3 Construction of Plastid-Targeting Vectors

The four expression cassettes mentioned above were all cloned into chloroplast-targeting plasmids in preparation for bombardment (see FIG. 7A). The chloroplast-targeting vectors contain regions of DNA homologous to the tobacco plastid genome (52377-59319, 59320-63864) that allow the target construct to be integrated by homologous replication in the plastid. The arrow in FIG. 7B highlights the position of DNA integration in the tobacco plastid genome (plastome).

The target gene construct is present in the vector, along with a selection agent expression cassette to promote stability of the transgene construct. aadA (aminoglycoside adenine transferase) detoxifies spectinomycin and streptomycin antibiotics, and is a preferred selection agent for use in accordance with the present invention.

Two regions of DNA homologous to the plastid genome flank the two expression cassettes. These regions direct homologous recombination to a specific region of the plastid genome. The flanking regions are known as the “left-” and “right-targeting regions” (LTR & RTR)

Flanking regions used insert the transgenic construct downstream of the extremely active rbcL gene, which produces the large subunit of rubsico—essential for photosynthesis.

### 2.4 Expression of Transgene Cassettes in E. Coli

Due to the prokaryotic origins of the plant plastid, chloroplast expression cassettes are often functional in bacteria such as Escherichia coli (E. coli). TGF-β3 protein expression was identified for each transgene construct in E. coli (data not shown). Total protein samples from E. coli were separated by SDS-PAGE, and Western blot analysis was carried out using antibodies specific to TGF-β3 protein.

As expression elements work in both bacteria and plastids, these studies are very useful at checking that expression cassettes are functional.

Western blots were carried out and TGF-β3 active region antibodies were used to check expression levels.

### 2.5 Transformation of N. Tabacum Plants

Wisconsin 38 (w38) tobacco leaves were transformed by particle bombardment followed by positive antibiotic selection to isolate clones. Shoots were grown on and rooted in MS media with antibiotics, and then plants were finally moved on to soil.

### 2.6 DNA Characterisation of Plants

Plants that were putative transformants had their DNA characterised by PCR and Southern Blot analysis to ascertain integration of the specific TGF-β3 gene and aadA marker gene (for antibiotic selection). Southern blot analysis confirmed correct integration of transgene cassettes and also confirmed homoplasmy in plants, which represents stable transformation.

### 2.7 Protein Characterisation

Leaf tissue from homoplasmic plants was harvested and analysed by SDS-PAGE and Western blot analysis. Expression of the TGF-β3 active region protein was identified by SDS-PAGE from the ‘16Srrn-T7-TGF-β3 active region-psbC’ and ‘165 mm-T7-TGF-β3 synthetic active region-psbC’ constructs; with protein expression quantified as ˜1% and ˜10% of total plant protein respectively (see FIG. 8) Quantification was carried out digitally with BioRad Quantity One software analysis on scanned gels. This result illustrates the great increase in yield that may be achieved using the methods and nucleic acids of the invention, in which nucleic acid sequences encoding TGF-βs are adapted for expression by plants.

Western blot analysis with TGF-β13 antibody confirmed the protein band of interest as TGF-β3 active region protein (see FIG. 9), and quantification of TGF-β3 standards confirmed that the protein expression levels mentioned above were correct.

Protein from the leaves of the ‘16Srrn-T7-TGF-β3 synthetic active region-psbC’ plant was prepared as either a soluble protein preparation or insoluble protein preparation and analysed by SDS-PAGE and Western blot (see FIG. 10). Results indicated that the synthetic TGF-β3 active region is expressed as an insoluble protein product.

### 3. Methods

### 3.1 Construction of the Synthetic TGF-β3 Active Region Gene

Coding regions from all twenty-nine chloroplast genes known to encode photosynthetic proteins have been analysed and tabulated as a codon usage table by Shimada et al (1991). The codon usage table was imported into the Vector NTI suite software (Informax) and the native TGF-β3 active region amino acid sequence was back-translated into a DNA coding region sequence. Where large numbers of a single codon-type existed, second or third most frequently used codons were included to reduce tRNA metabolic load and/or reduce repeating sequence. The resultant DNA sequence represented the optimised synthetic TGF-β3 active region for expression in N. tabacum chloroplasts.

The 350 bp synthetic TGF-β3 active region DNA coding region was assembled from single-stranded oligonucleotides using a step-wise construction process (see FIG. 3A). Oligonucleotide overlap, Klenow enzyme-directed DNA base fill-in, Vent- polymerase-mediated single stranded (ss) DNA production, and double-stranded (ds) DNA PCR amplification techniques were used to promote assembly of the synthetic construct. FIG. 3B shows an agarose gel representing construction progress of the synthetic gene. dsDNA molecules of˜35, 60, 100, 180, 225 and 350 bp can be seen on the gel, which represent the gene fragments being assembled stepwise. The final 350 bp construct was A-tailed, cloned into the pGEM-T vector (Invitrogen) and sequenced to confirm sequence integrity.

### 3.2 Plastid Transformation of Tobacco

### 3.2.1 Preparation of Leaves

Wisconsin 38 (W38) tobacco was grown for 5 weeks from seed on MS media with sucrose. At this stage plants with approximately 4-6 medium sized leaves were present in growth vessels. These leaves were cut at the base of the leaf tissue and placed abaxial side up, in the centre of RMOP plates. Plates were covered, sealed and placed in a growth cabinet until required for DNA bombardment.

### 3.2.2 Preparation of DNA-Coated Microcarriers

Gold particles (1.0 μm diameter, BioRad) were washed in ethanol by vortexing. These microcarriers were centrifuged and the supernatant removed, before adding s.d.H₂O and vortexing briefly again. Aliquots of this gold solution were transferred to 1.5 ml centrifuge tubes. Targeting plasmid DNA was added to the microcarrier suspension aliquots and vortexed briefly. 2.5M CaCl₂ was immediately added to the gold preparation while mixing, and this was followed quickly by addition of 0.1M spermidine. The microcarrier preparation was vortexed and centrifuged. The supernatant was removed and the microcarriers washed with EtOH by vortexing. The microcarriers were again centrifuged and the supernatant removed. Microcarriers were re-suspended in EtOH by briefly vortexing. Sterile macrocarrier discs were placed into metal-holding plates and aliquots of the microcarrier preparation were pipetted onto the centre of each macrocarrier. The microcarrier solution evaporated to leave a small circular precipitate on the macrocarrier surface. At this point macrocarriers were ready for bombardment experiments.

### 3.2.3 Particle Bombardment

Particle bombardment of tobacco leaves was carried out using Bio-Rad gene gun apparatus in a laminar flow hood. Set-up of the apparatus, production of the vacuum and gas release steps were carried out according to the manufacturers instructions. The leaf tissue is placed in the lower section of the compartment, with the lid of the plate removed. Microcarriers containing DNA vectors are accelerated into the plant tissue. 1100 psi rupture discs were used and a projectile distance of 10 cm between the stopping screen and plant tissue employed. After each particle bombardment, plates with tobacco leaves were re-covered, sealed, and incubated in a growth cabinet at 23° C. for 48 hrs, with a 12 hr light/dark cycle. Light intensity was˜150 μEi.

### 3.2.4 Post-Bombardment Leaf Selection

48 hrs post-bombardment, leaf tissue was cut into˜2 mm² pieces, and placed onto selective media. This selective media was either RMOP with 500 μg/ml spectinomycin, or RMOP with 500 μg/ml spectinomycin plus 250 μg/ml streptomycin. Tissue plates were incubated at 23° C., in a 12 hr light/dark cycle with light intensity of ˜150 μEi. Transformed cells regenerated as plant shoots between 4-8 weeks, and were transferred into growth vessels with MS media plus 250 μg/ml spectinomycin to grow and root. Putative transformants were screened for transgenes using PCR and then their DNA characterised by Southern blot anlaysis.

### 3.3 DNA Characterisation

DNA analysis was carried out by first harvesting plant leaves and grinding in liquid nitrogen. DNA was prepared using the Eppendorf ‘plant DNA prep’ kit. DNA samples were cleaved by restriction enzyme digest and size-separated by gel-electrophoresis. DNA was transferred to nylon membranes and then hybridised with ³²P-dCTP labelled DNA probes to identify TGF-β3 genes, marker genes and native chloroplast genes. Probe hybridisation identified integrated genes, and restriction digest patterns allowed for DNA integration maps to be confirmed.

### 3.4 Protein Characterisation

### 3.4.1 SDS-PAGE Analysis

For total cellular protein preparations, leaf tissue was ground to a powder in liquid nitrogen and added in a 1:5 ratio (w/v) to 1× sample buffer. Samples were placed in a boiling water bath for 5 mins, then centrifuged. The supernatant was then collected and used for SDS-PAGE analysis. For soluble cellular protein preparations, ground frozen leaf tissue was vortexed and incubated in extraction buffer and then centrifuged to remove solids. The supernatant was isolated and its protein content quantified. Soluble protein samples were added to 2× Sample buffer and placed in a boiling water bath for 5 mins. Samples were centrifuged and the supernatant collected for SDS-PAGE analysis. For insoluble protein preparations, the pellet that remained from the soluble protein extract was re-suspended and washed 3 times in extraction buffer, centrifuging after each wash. The remaining pellet was then re-suspended in 1× Sample buffer, placed in a boiling water bath for 5 mins, then centrifuged and the supernatant collected for SDS-PAGE analysis. 10-20% Tris-HCl acrylamide gel electrophoresis was used to separate proteins by size, with protein bands visualised by Coomassie blue staining.

### 3.4.2 Western Blot Analysis

Protein samples were separated by size on SDS-PAGE gels and then transferred to nylon membranes. Membranes were blocked, probed with TGF-β3 antibody and then washed. TGF-β3 protein was visualised by BCIP staining of the alkaline-phosphatase linked antibody.

### EXPERIMENTAL RESULTS II

### 4 Recovery of Expressed TGF-β3

TGF-β3 expressed in plant chloroplasts using the techniques described above was recovered using the technique described for the first time below. This technique produce higher yields of TGF-β, and TGF-β having greater purity, than recovery or purification techniques described in the prior art.

Chloroplast extracts were diluted 1:1 in lysis buffer (comprising 10 mM HEPES, 5 mM EDTA, 2% weight/weight Triton X-100, 0.1 M DTT at pH 8.0). This mixture was homogenized and sonicated to aid dissolution. The resultant solution was then centrifuged at 8000×g for 30 minutes.

The pellet produced on centrifugation above was re-suspended to the original volume using a wash buffer (comprising 0.05M Tris base, 0.01 M EDTA at pH 8.0), before a further round of centrifugation at 8000×g for 30 minutes.

The pellet produced by this round of centrifugation was washed and then re-suspended in solubilisation buffer (comprising 0.05M Tris base, 0.1 M DTT, 6M Urea at pH 8.0) to give rise to a ten fold dilution (i.e. one volume of the pellet material added to nine volumes of the solubilisation buffer). The resulting solution was stirred for 60 minutes at room temperature to solubilise the re-suspended material. After 60 minutes of stirring the pH of the solubilised solution was adjusted to 9.5, and stirring continued for a further 60 minutes at room temperature.

The pH-adjusted solution was then centrifuged at 8000×g for 30 minutes, during which time a process of diafiltration using a 5 kDa TFF (tangential flow filtration) membrane was used to exchange the diluent to a diafiltration buffer (0.05 M Tris base, 0.01 M DTT, 3 M Urea at pH 9.5), and to concentrate the solutions so produced by 15 fold. This concentrated solution (the retentate) was then subject to re-folding using the conditions described below.

### Analysis of Recovered TGF-β3

The presence of TGF-β3 in the solution to be re-folded was confirmed using a Biorad RC/DC assay. The results of this are shown in FIG. 11. FIG. 11 shows results achieved using a 12% Bis-Tris Reduced Gel in which protein has been labelled with Coomassie Blue. The lanes (1-10 reading from left to right) were loaded with samples as follows:

### Lane 1=Mark 12 Standard

### Lane 2=TGF-β Standard

Lane 3=Lysed material
Lane 4=Lysed material supernatant
Lane 5=Wash supernatant
Lane 6=Solubilised supernatant
Lane 7=Solubilised supernatant
Lane 8=Solubilised supernatant

### Lane 9=Blank

Lane 10=Solubilised supernatant

These results confirm that TGF-β3 expressed using the methods of the invention may be obtained from lysed chloroplast material, and that using the recovery regime outlined above this material may be concentrated in the solubilised supernatant prior to re-folding.

### 6 Re-Folding of Expressed TGF-β3

The material described above was diluted into a re-folding buffer (comprising 0.7 M CHES, 1 M NaCl, 0.002 M reduced glutathione, 0.0004M oxidised glutathione, 0.25 mg/mL TGF-β3 monomer expressed in accordance with the invention, all at pH 9.5) this re-folding mixture was then maintained, with stirring, at 10° C. for 3 days to allow re-folding to occur. This re-folding procedure, conducted in the presence of 2-(cyclohexylamino)ethanesulfonic acid (CHES) was found by the inventors to produce a particularly high yield of correctly folded TGF-β3. Accordingly the folding (or re-folding) of TGF-βs expressed in accordance with the invention in the presence of CHES represents a particularly useful and advantageous embodiment of the present invention.

### 7 Capture of Re-Folded TGF-β13 Expressed in Accordance with the Invention

Re-folded TGF-β3 produced as described as above, was concentrated five fold in a preconditioned UF system fitted with a membrane with a MWCO of 5 kDa. The pH of the refold concentrate was adjusted stepwise from pH 2.5 to 2.8 using glacial acetic acid. The acidified concentrate was then diluted in a ratio of 1:1 using Dilution Buffer (0.02 M sodium acetate, 2 M ammonium sulphate, 1 M arginine hydrochloride, 8.33% (w/w) acetic acid) and filtered through a 0.22 μm filter. This “conditioned load” was added to a Butyl-Sepharose 4 Fast Flow separation medium in order to capture the re-folded TGF-β3 by hydrophobic interaction chromatography. The Butyl-Sepharose 4 Fast Flow column was equilibrated with wash buffer/equilibration buffer (comprising 0.02 M Sodium Acetate, 1 M Ammonium Sulphate, 10% volume for volume Acetic Acid, at pH 3.3). The column was washed with four column volumes (CVs) of this equilibration buffer prior to step elution of bound TGF-β3. Step elution was conducted using an elution buffer (comprising 0.02 M Sodium Acetate, 10% volume for volume Acetic Acid, 30% volume for volume Ethanol at pH 3.3) and the TGF-β3 eluates produced in this manner pooled.

Analysis of the purified TGF-β3 produced in the pooled eluates is shown in FIG. 12, which illustrates that TGF-β3 expressed in plants using the methods of the invention may be purified to yield re-folded TGF-β3 using the methods described herein. It will be appreciated that these methods may also be used in the recovery, re-folding and capture of biologically active TGF-βs other than TGF-β3. Purification of the biologically active TGF-β3 produced using the methods described above may alternatively or additionally be carried out using the following procedure.

### 8 Purification of TGF-β3 Expressed in Accordance with the Invention

In an alternative purification process, the eluate from the Butyl-Sepharose capture purification step was pH adjusted to 4.0 (±0.1) and diluted with a buffer comprising 2.72 g/L sodium acetate trihydrate, 100 mL/L glacial acetic acid and 300 mL/L ethyl alcohol at pH 3.9-4.1) until the conductivity met the required specification of <7.0 mS/cm. The conditioned Butyl eluate was then filtered through a 0.22 μm filter before it was loaded onto a SP-Sepharose column equilibrated with wash buffer and equilibration buffer comprising: 2.72 g/L sodium acetate trihydrate, 100 mL/L glacial acetic acid, 300 mL/L ethyl alcohol and 2.92 g/L sodium chloride at pH 3.9-4.1. The column was then washed with 3 column volumes of wash buffer and equilibration buffer. A linear gradient 0% to 50% of Elution Buffer (2.72 g/L sodium acetate trihydrate, 100 mL/L glacial acetic acid, 300 mL/L ethyl alcohol, 29.22 g/L sodium chloride at pH 3.9-4.1) was applied to the column over fifteen column volumes. The column was then washed with a step gradient of 50% to 100% of Elution Buffer, followed by 2-3 column volumes of 1 M sodium chloride. Fractions of the SP Sepharose eluate containing TGF-β3 dimer were pooled according to purity by RP-HPLC. The pooled SP-Sepharose eluate was concentrated to a TGF-β3 concentration of 12 mg/mL (by A₂₇₈ₙₘ) using a preconditioned UF/DF system (with a MWCO of 5 kDa). The concentrated TGF-β3 solution was then buffer exchanged into the Formulation Buffer (1.2 mL/L acetic acid, 200 mL/L ethyl alcohol at pH 4.0±0.1) over 6 diavolumes. The diafiltered TGF-β3 solution was then diluted to a TGF-β3 concentration of 10±2 mg/mL(by A₂₇₈ₙₘ) with the Formulation Buffer.

### Sequence Information

Amino acid sequence of active fragment of TGF-β 1
(Sequence ID No. 1)
ALDTNYCFSSTEKNCCVRQLYIDFRKDLGWKWIHEPKGYHANFCLGPCPYIWSLDT
QYSKVLALYNQHNPGASAAPCCVPQALEPLPIVYYVGRKPKVEQLSNMIVRSCKCS
Amino acid sequence of active fragment of TGF-β 2
(Sequence ID No. 2)
ALDAAYCFRNVQDNCCLRPLYIDFKRDLGWKWTHEPKGYNANFCAGACPYLWSSDT
QHSRVLSLYNTINPEASASPCCVSQDLEPLTILYYIGKTPKIEQLSNMIVKSCKCS
Amino acid sequence of active fragment of TGF-β 3
(Sequence ID No. 3)
ALDTNYCFRNLEENCCVRPLYIDFRQDLGWKWVHEPKGYYANFCSGPCPYLRSADT
THSTVLGLYNTLNPEASASPCCVPQDLEPLTILYYVGRTPKVEQLSNMVVKSCKCS
Native DNA sequence encoding active fragment of TGF-β 3
(Sequence ID No. 4)

ATGGCTTTGGACACCAATTACTGCTTCCGCAACTTGGAGGAGAACTGCTGTGTGCGCCCCCTCTACATTGAC
TTCCGACAGGATCTGGGCTGGAAGTGGGTCCATGAACCTAAGGGCTACTATGCCAACTTCTGCTCAGGCCCT
TGCCCATACCTCCGCAGTGCAGACACAACCCACAGCACGGTGCTGGGACTGTACAACACTCTGAACCCTGAA
GCATCTGCCTCGCCTTGCTGCGTGCCCCAGGACCTGGAGCCCCTGACCATCCTGTACTATGTTGGGAGGACC
CCCAAAGTGGAGCAGCTCTCCAACATGGTGGTGAAGTCTTGTAAATGTAGCTGA
Second nucleic acid sequence of the invention encoding active
fragment of TGF-β 3

(Sequence ID No. 5)

ATGGCTTTAGATACTAATTATTGTTTTCGTAATTTAGAAGAAAATTGTTGCGTACGTCCTTTATATATTGAT
TTTCGTCAAGATCTTGGTTGGAAATGGGTACATGAACCTAAAGGTTATTATGCTAATTTTTGTTCTGGTCCT
TGTCCTTATTTGCGTTCTGCTGATACTACTCATTCTACTGTTTTAGGTCTTTATAATACTTTAAATCCTGAA
GCATCTGCTAGTCCTTGTTGCGTACCTCAAGATTTGGAACCTTTAACTATTCTTTATTACGTAGGTCGTACT
CCTAAAGTTGAACAATTGTCTAACATGGTAGTTAAAAGTTGTAAATGTTCTTAA

| DNA encoding full-length TGF-Beta 1, showing signal peptide | | |
|---|---|---|
| (shown in italics), pro-peptide (shown in bold) as well as the active | | |
| fragment (shown in normal text) | | |
| (Sequence ID No. 6) | | |
| 60 | atgccgccct ccgggctgcg gctgctgctg ctgctgctac cgctgctgtg gctactggtg | |
| | ctgacgcctg gccggccggc cgcgggacta tccacctgca agactatcga catggagctg | 120 |
| | gtgaagcgga agcgcatcga ggccatccgc ggccagatcc tgtccaagct gcggctcgcc | 180 |
| | agccccccga gccaggggga ggtgccgccc ggcccgctgc ccgaggccgt gctcgccctg | 240 |
| | tacaacagca cccgcgaccg ggtggccggg gagagtgcag aaccggagcc cgagcctgag | 300 |
| | gccgactact acgccaagga ggtcacccgc gtgctaatgg tggaaaccca caacgaaatc | 360 |
| | tatgacaagt tcaagcagag tacacacagc atatatatgt tcttcaacac atcagagctc | 420 |
| | cgagaagcgg tacctgaacc cgtgttgctc tcccgggcag agctgcgtct gctgaggctc | 480 |
| | aagttaaaag tggagcagca cgtggagctg taccagaaat acagcaacaa ttcctggcga | 540 |
| | tacctcagca accggctgct ggcacccagc gactcgccag agtggttatc ttttgatgtc | 600 |
| | accggagttg tgcggcagtg gttgagccgt ggaggggaaa ttgagggctt tcgccttagc | 660 |
| | gcccactgct cctgtgacag cagggataac acactgcaag tggacatcaa cgggttcact | 720 |
| | accggccgcc gaggtgacct ggccaccatt catggcatga accggccttt cctgcttctc | 780 |
| | atggccaccc cgctggagag ggcccagcat ctgcaaagct cccggcaccg ccgagccctg | 840 |
| | gacaccaact attgcttcag ctccacggag aagaactgct gcgtgcggca gctgtacatt | 900 |
| | gacttccgca aggacctcgg ctggaagtgg atccacgagc ccaagggcta ccatgccaac | 960 |
| | ttctgcctcg ggccctgccc ctacatttgg agcctggaca cgcagtacag caaggtcctg | 1020 |
| | gccctgtaca accagcataa cccgggcgcc tcggcggcgc cgtgctgcgt gccgcaggcg | 1080 |
| | ctggagccgc tgcccathgt gtactacgtg ggccgcaagc ccaaggtgga gcagctgtcc | 1140 |
| | aacatgatcg tgcgctcctg caagtgcagc tga | 1173 |
| DNA encoding full-length TGF-Beta 2, showing signal peptide | | |
| (shown in italics), pro-peptide (shown in bold) as well as the active | | |
| fragment (shown in normal text) | | |
| (Sequence ID No. 7) | | |
| 60 | ctgtctacct gcagcacact cgatatggac cagttcatgc gcaagaggat [symbol] | 120 |
| | cgcgggcaga tcctgagcaa gctgaagctc accagtcccc cagaagacta tcctgagccc | 180 |
| | gaggaagtcc ccccggaggt gatttccatc tacaacagca ccagggactt gctccaggag | 240 |
| | aaggcgagcc ggagggcggc cgcctgcgag cgcgagagga gcgacgaaga gtactacgcc | 300 |
| | aaggaggttt acaaaataga catgccgccc ttcttcccct ccgaagccat cccgcccact | 360 |
| | ttctacagac cctacttcag aattgttcga tttgacgtct cagcaatgga gaagaatgct | 420 |
| | tccaatttgg tgaaagcaga gttcagagtc tttcgtttgc agaacccaaa agccagagtg | 480 |
| | cctgaacaac ggattgagct atatcagatt ctcaagtcca aagatttaac atctccaacc | 540 |
| | cagcgctaca tcgacagcaa agttgtgaaa acaagagcag aaggcgaatg gctctccttc | 600 |
| | gatgtaactg atgctgttca tgaatggctt caccataaag acaggaacct gggatttaaa | 660 |
| | ataagcttac actgtccctg ctgcactttt gtaccatcta ataattacat catcccaaat | 720 |
| | aaaagtgaag aactagaagc aagatttgca ggtattgatg gcacctccac atataccagt | 780 |
| | ggtgatcaga aaactataaa gtccactagg aaaaaaaaca gtgggaagac cccacatctc | 840 |
| | ctgctaatgt tattgccctc ctacagactt gagtcacaac agaccaaccg gcggaagaag | 900 |
| | cgtgctttgg atgcggccta ttgctttaga aatgtgcagg ataattgctg cctacgtcca | 960 |
| | ctttacattg atttcaagag ggatctaggg tggaaatgga tacacgaacc caaagggtac | 1020 |
| | aatgccaact tctgtgctgg agcatgcccg tatttatgga gttcagacac tcagcacagc | 1080 |
| | agggtcctga gcttatataa taccataaat ccagaagcat ctgcttctcc ttgctgcgtg | 1140 |
| | tcccaagatt tagaacctct aaccattctc tactacattg gcaaaacacc caagattgaa | 1200 |
| | cagctttcta atatgattgt aaagtcttgc aaatgcagct aa | 1242 |

| DNA encoding full-length TGF-Beta 3, showing signal peptide | |
|---|---|
| (shown in italics), pro-peptide (shown in bold) as well as the | |
| active fragment (shown in normal text) | |
| (Sequence ID No. 8) | |
| atgaagatgc acttgcaaag ggctctggtg gtcctggcca tgctgaactt tgccacggtc | 60 |
| agcctctctc tgtccacttg caccaccttg gacttcggcc acatcaagaa gaagagggtg | 120 |
| gaagccatta ggggacagat cttgagcaag ctcaggctca ccagcccccc tgagccaacg | 180 |
| gtgatgaccc acgtccccta tcaggtcctg gccctttaca acagcacccg ggagctgctg | 240 |
| gaggagatgc atggggagag ggaggaaggc tgcacccagg aaaacaccga gtcggaatac | 300 |
| tatgccaaag aaatccataa attcgacatg atccaggggc tggcggagca caacgaactg | 360 |
| gctgtctgcc ctaaaggaat tacctccaag gttttccgct tcaatgtgtc ctcagtggag | 420 |
| aaaaatagaa ccaacctatt ccgagcagaa ttccgggtct tgcgggtgcc caaccccagc | 480 |
| tctaagcgga atgagcagag gatcgagctc ttccagatcc ttcggccaga tgagcacatt | 540 |
| gccaaacagc gctatatcgg tggcaagaat ctgcccacac ggggcactgc cgagtggctg | 600 |
| tcctttgatg tcactgacac tgtgcgtgag tggctgttga gaagagagtc caacttaggt | 660 |
| ctagaaatca gcattcactg tccatgtcac acctttcagc ccaatggaga tatcctggaa | 720 |
| aacattcacg aggtgatgga aatcaaattc aaaggcgtgg acaatgagga tgaccatggc | 780 |
| cgtggagatc tggggcgcct caagaagcag aaggatcacc acaaccctca tctaatcctc | 840 |
| atgatgattc ccccacaccg gctcgacaac ccgggccagg ggggtcagag gaagaagcgg | 900 |
| gctttggaca ccaattactg cttccgcaac ttggaggaga actgctgtgt gcgccccctc | 960 |
| tacattgact tccgacagga tctgggctgg aagtgggtcc atgaacctaa gggctactat | 1020 |
| gccaacttct gctcaggccc ttgcccatac ctccgcagtg cagacacaac ccacagcacg | 1080 |
| gtgctgggac tgtacaacac tctgaaccct gaagcatctg cctcgccttg ctggctgccc | 1140 |
| caggacctgg agcccctgac catcctgtac tatgttggga ggacccccaa agtggagcag | 1200 |
| ctctccaaca tggtggtgaa gtcttgtaaa tgtagctga | |

1. A method for the expression of a TGF-β in a plant, said method comprising:
(a) introducing into a plant cell a chimeric nucleic acid sequence comprising:
(1) a first nucleic acid sequence capable of regulating the transcription in a plant cell of
(2) a second nucleic acid sequence, encoding a TGF-β, and adapted for expression in the plant cell; and
(3) a third nucleic acid sequence encoding a termination region functional in said plant cell; and
(b) growing said plant cell to produce said TGF-β.

2. A method according to claim 1, wherein the nucleic acid sequence is selected from the group consisting of: a nucleic acid sequence suitable to be expressed in a chloroplast of a plant cell and a nucleic acid sequence adapted to be expressed in a chloroplast of a plant cell.

3. (canceled)

4. A method according to claim 1, wherein the TGF-β is a human TGF-β.

5. A method according to claim 1, wherein the TGF-β is TGF-β3.

6. A method according to claim 1, wherein the TGF-β comprises a TGF-β active fragment selected from the group consisting of: Sequence ID No. 1; Sequence ID No. 2; and Sequence ID No. 3.

7. A method according to claim 1, wherein the TGF-β comprises the full length TGF-β protein.

8. A method according to claim 1, wherein the TGF-β comprises a TGF-β proprotein.

9. A method according to claim 1, wherein the second nucleic acid sequence comprises at least one substitution selected from the group consisting of:
a UGC codon compared to the native DNA encoding the TGF-β3 ;
a CUG codon compared to the native DNA encoding the TGF-β;
a UAC codon compared to the native DNA encoding the TGF-β;
a GUG codon compared to the native DNA encoding the TGF-β;
a CCC codon compared to the native DNA encoding the TGF-β;
a AAC codon compared to the native DNA encoding the TGF-β; and
a GAC codon compared to the native DNA encoding the TGF-β.

10.-15. (canceled)

16. A method according to claim 1, wherein the first nucleic acid sequence comprises a plastid promoter selected from the group consisting of: promoters expressing photosynthesis-related genes; promoters expressing genetic system genes; promoters expressing genes recognised by the plastid encoded plastid (PEP) RNA polymerase or nucleus-encoded plastid (NEP) RNA polymerase; a plastid psbA promoter; and a plastid 16S rrn promoter.

17. A method according to claim 1, wherein the first nucleic acid sequence comprises a promoter selected from the group consisting of: a Chlamydomonas psbA promoter; a bacterial trc promoter; a bacteriophage T7 promoter; and a 16srrn promoter.

18.-20. (canceled)

21. A method according to claim 1, wherein the first nucleic acid sequence comprises a ribosome binding site (RBS) selected from the group consisting of:
i) a plastid RBS;
ii) a bacterial RBS; and
iii) a bacteriophage RBS.

22. (canceled)

23. A method according to claim 1, wherein the third nucleic acid sequence comprises a terminator selected from the group consisting of:
i) a plastid terminator;
ii) a bacterial terminator; and
iii) a bacteriophage terminator.

24. (canceled)

25. A method according to claim 1, wherein the chimeric nucleic acid sequence further comprises a nucleic acid sequence for selection of transformed cells.

26. A method according to any of claim 1, wherein the second nucleic acid sequence comprises Sequence ID No. 5, or a sequence having at least 22% codon identity with Sequence ID No. 5.

27. (canceled)

28. A method according to claim 1, further comprising dissolving the TGF-β in a solvent capable of preferentially solubilising recombinant TGF-β, but not plant cell components.

29. (canceled)

30. A method according to any of claim 1, further comprising diafiltration to concentrate a solution of the TGF-β.

31. A method according to claim 1, further comprising folding the TGF-β in the presence of CHES (2-(cyclohexylamino)ethanesulfonic acid), or a functional analogue thereof, such that active TGF-β is produced.

32. (canceled)

33. A method according to claim 1, further comprising using the TGF-β so expressed in the manufacture of a medicament.

34. A method according to claim 33, wherein the medicament is for the prevention of scarring or fibrosis.

35. A TGF-β produced by the method of claim 1.

36. A TGF-β according to claim 35, wherein the TGF-β is TGF-β3.

37. A TGF-β according to claim 35, wherein the TGF-β comprises a TGF-β active fragment selected from the group consisting of: Sequence ID No. 1;
Sequence ID No. 2; and Sequence ID No. 3.

38. A TGF-β according to claim 35, wherein the TGF-β comprises a TGF-β proprotein.

39. A chimeric nucleic acid sequence comprising:
(1) a first nucleic acid sequence capable of regulating the transcription in a plant cell of
(2) a second nucleic acid sequence, encoding a TGF-β, and adapted for expression in a plant cell; and
(3) a third nucleic acid sequence encoding a termination region functional in a plant cell.

40. The nucleic acid of claim 39, comprising a nucleic acid sequence selected from the group consisting of: a nucleic acid sequence suitable to be expressed in a chloroplast of a plant cell and a nucleic acid sequence adapted to be expressed in a chloroplast of a plant cell.

41. (canceled)

42. A nucleic acid sequence according to claim 39, comprising a nucleic acid sequence of Sequence ID No. 5, or a sequence having at least 22% codon identity with Sequence ID No. 5.

43. A plant transformed with a nucleic acid according to claim 39.

44. A plant seed comprising a nucleic acid according to claim 39.

45. A medicament comprising a TGF-beta produced in accordance with claim 1.


SEQUENCE LISTING (8 sequences)

SEQ ID NO: 1 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 3 - protein, 112 aa, Homo sapiens [sequence omitted]

SEQ ID NO: 4 - DNA, 342 bp, Homo sapiens
atggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctga

SEQ ID NO: 5 - DNA, 342 bp, Artificial
  Second nucleic acid sequence of the invention encoding active fragment of TGF-beta 3
atggctttagatactaattattgttttcgtaatttagaagaaaattgttgcgtacgtcctttatatattgattttcgtcaagatcttggttggaaatgggtacatgaacctaaaggttattatgctaatttttgttctggtccttgtccttatttgcgttctgctgatactactcattctactgttttaggtctttataatactttaaatcctgaagcatctgctagtccttgttgcgtacctcaagatttggaacctttaactattctttattacgtaggtcgtactcctaaagttgaacaattgtctaacatggtagttaaaagttgtaaatgttcttaa

SEQ ID NO: 6 - DNA, 1173 bp, Homo sapiens
atgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccathgtgtactacgtgggccgcaagcccaaggtggagcagctgtccaacatgatcgtgcgctcctgcaagtgcagctga

SEQ ID NO: 7 - DNA, 1182 bp, Homo sapiens
ctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaagccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 8 - DNA, 1239 bp, Homo sapiens
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccgcaacttggaggagaactgctgtgtgcgccccctctacattgacttccgacaggatctgggctggaagtgggtccatgaacctaagggctactatgccaacttctgctcaggcccttgcccatacctccgcagtgcagacacaacccacagcacggtgctgggactgtacaacactctgaaccctgaagcatctgcctcgccttgctgcgtgccccaggacctggagcccctgaccatcctgtactatgttgggaggacccccaaagtggagcagctctccaacatggtggtgaagtcttgtaaatgtagctga